(19) United States
(12) Patent Application Publication
(10) Pub. No.: US 2020/0000695 A1
(43) Pub. Date: Jan. 2, 2020

(54) COSMETIC OR DERMATOLOGICAL PREPARATION WITH A CONTENT OF CREATINE, CREATININE OR DERIVATIVES THEREOF IN COMBINATION WITH SOYBEAN GERM EXTRACT

(71) Applicants: Franz STAEB, Echem (DE); Thomas BLATT, Wedel (DE); Melanie SCHMIDT, Hamburg (DE); Claudia MUNDT, Bremen (DE); Stefan GALLINAT, Wedel (DE); Kirsten VENZKE, Hamburg (DE); Holger LENZ, Hamburg (DE); Cornelia MEIER-ZIMMERER, Hamburg (DE)

(72) Inventors: Franz STAEB, Echem (DE); Thomas BLATT, Wedel (DE); Melanie SCHMIDT, Hamburg (DE); Claudia MUNDT, Bremen (DE); Stefan GALLINAT, Wedel (DE); Kirsten VENZKE, Hamburg (DE); Holger LENZ, Hamburg (DE); Cornelia MEIER-ZIMMERER, Hamburg (DE)

(73) Assignee: BEIERSDORF AG, Hamburg (DE)

(21) Appl. No.: 16/542,434

(22) Filed: Aug. 16, 2019

Related U.S. Application Data
(62) Division of application No. 11/183,376, filed on Jul. 18, 2005.
Application No. 11/183,376 is a continuation of application No. PCT/EP04/50015, filed on Jan. 14, 2004.

(30) Foreign Application Priority Data
Jan. 17, 2003 (DE) 10301632.5

(51) Int. Cl. A61K 8/49

(52) U.S. Cl. CPC A61K 8/4946

(57) ABSTRACT

A cosmetic or dermatological preparation comprising (a) at least one creatinine compound; (b) at least one creatine compound; and (c) at least one soybean substance selected from soybean germ extracts and ingredients which can be isolated from soybean germ are used in methods for stimulating collagen synthesis of the skin and for restructuring or rejuvenation of the skin.

### CROSS-REFERENCE TO RELATED APPLICATIONS

This application is a divisional of application Ser. No. 11/183,376, which is a continuation of application PCT/EP04/050015, filed Jan. 14, 2004, incorporated by reference herein in their entireties, and also claims the benefit of German Patent Application No. 103 01 632.5, filed Jan. 17, 2003.

### FIELD OF THE INVENTION

The present invention concerns the use of combinations of creatinine and/or creatinine derivates with creatine and/or its derivatives with soybean germ extracts or ingredients which can be isolated from this, preferably daidzin, glycitin, genistin, daidzein, glycitein, genistein as well as saponins in cosmetic or dermatological preparations for the treatment and prophylaxis of the symptoms of UV-induced or ozone-induced skin damage as well as inflammatory and degenerative skin conditions.

### BACKGROUND OF THE INVENTION

Cosmetic skincare primarily involves treatment and prophylaxis, whereby the natural function of the skin is strengthened or restored as a barrier against environmental influences (e.g. dirt, chemicals, microorganisms) and against the loss of the body's own substances (e.g. water, natural fats, electrolytes).

If this function is damaged, the result can be increased resorption of toxic or allergenic substances or attack by microorganisms and consequently toxic or allergenic skin reactions.

The aim of skin care is moreover to compensate for the loss of fat and water in the skin caused through daily washing. This is particularly important if the natural regeneration capacity is not sufficient. Furthermore, skincare products should also offer protection against environmental influences, in particular sun and wind, and slow the skin ageing process.

Chronological skin ageing is typically caused by endogenous, genetically-determined factors. In the epidermis and dermis, the ageing process causes the following structural damage and functional disorders, which can be summarized under the term “senile xerosis”:

a) Dryness, roughness and the formation of dryness wrinkles,

b) Itchiness and

c) Reduced fat restoration by the sebaceous glands (e.g. after washing).

Exogenous factors, such as UV light and noxious chemicals, can have a cumulative effect and typically accelerate the endogenous ageing process or contribute to it. In the epidermis and dermis, this typically leads to the following structural damage and functional disorders in the skin as a result of exogenous factors in particular, which extend beyond the degree and nature of the damage occurring in chronological ageing:

d) Visible vessel dilations (telangiectasias, cuperosis),

e) flabbiness and the formation of wrinkles;

f) Local hyper-, hypo- and malpigmentation (e.g. age marks) and

g) Increased susceptibility to mechanical stress (e.g. crack formation).

The present invention concerns, in particular, products for the care of skin which has aged naturally, as well as treatment of the consequential damage resulting from light ageing, in particular the phenomena cited under a) to g).

Products for the care of aged skin are familiar per se. They contain, for instance, retinoids (vitamin A acid and derivates thereof) or vitamin A and derivatives thereof. Their effect on structural damage is nevertheless restricted in terms of extent. Furthermore, there are considerable difficulties in product development when it comes to stabilizing the active agents against oxidative decomposition to a sufficient degree. The use of products containing vitamin A acid also frequently causes strongly erythematous skin irritations. Retinoids can therefore only be used in low concentrations.

In particular, the present invention concerns cosmetic preparations with an effective protection against harmful oxidation processes in the skin, but also as protection for cosmetic preparations themselves or as protection for the components of cosmetic preparations against oxidation processes.

The harmful effects of the ultraviolet component of sunlight on the skin are universally known. While radiation with a wavelength less than 290 nm (the so-called UVC region) is absorbed by the ozone layer in the earth's atmosphere, radiation in the range between 290 nm and 320 nm, the so-called UVB range, causes an erythema, straightforward sunburn or even more or less significant burns. The narrow range around 308 nm in sunlight is cited as having the maximum erythema-inducing effect.

Numerous compounds are familiar as protection against UVB radiation. These involve derivatives of 3-benzylidene camphor, 4-aminobenzoic acid, cinnamic acid, salicylic acid, benzophenone as well as 2-phenylbenzimidazole.

For the region between around 320 nm and around 400 nm, the so-called UVA region, it is also important to have filter substances available, as its radiation can cause reactions in light-sensitive skin. It has been demonstrated that UVA radiation causes damage to the elastic and collagenous fibers of the connective tissue, which can lead to the skin ageing prematurely. It is therefore regarded as the cause of numerous phototoxic and photoallergic reactions. The harmful effects of UVB radiation can be intensified by UVA radiation.

Certain derivatives of dibenzoylmethane are therefore used as protection against radiation in the UVA region, the photostability of which is not sufficiently indicated (Int. J. Cosm. Science 10, 53 (1988)).

UV radiation can, however, also lead to photochemical reactions, whereby the photochemical reaction products interfere with the metabolism of the skin.

Such photochemical reaction products primarily involve radical compounds, for example hydroxyl radicals. Undefined radical photoproducts which arise in the skin itself can also induce uncontrolled consequential reactions on account of their high degree of reactivity. Moreover, singlet oxygen, a non-radical excited state of oxygen molecules, can also occur in the event of exposure to UV radiation, as can short-lived epoxides and many other chemicals. Singlet oxygen is typically characterized by increased reactivity in comparison to the normally occurring state of triplet oxygen (radical basic state). Nevertheless, excited, reactive (radical) triplet states of the oxygen molecule also exist.

Furthermore, UV radiation also represents ionizing radiation. There is also therefore a risk that ionic species will also arise on exposure to UV radiation, which for their part can then interfere oxidatively with the biochemical processes.

In order to prevent these reactions, additional antioxidants and/or radical interceptors can be incorporated in the cosmetic or dermatological formulations.

The use of vitamin E, a substance with a well-known antioxidant effect, in light protection formulations has already been proposed, however the effect attained by this remains far behind what had been hoped for.

The task of the invention was therefore also to create cosmetic, dermatological and pharmaceutical substances and preparations as well as light protection formulations, which serve for the prophylaxis and treatment of light-sensitive skin, in particular photodermatoses, preferably PLD.

Further designations for polymorphous light dermatosis are PLD, PLE, Mallorca acne and a variety of further names, as indicated in the specialist literature (e.g. A. Voelckel et al, Zentralblatt Haut- and Geschlechtskrankheiten (1989), 156, P. 2).

Antioxidants are primarily used as protective substances against deterioration of the preparations containing them. Nevertheless, it is known that unwanted oxidation processes can also occur in human and animal skin. Such processes play a significant role in skin ageing.

The paper “Skin Diseases Associated with Oxidative Injury” in “Oxidative Stress in Dermatology”, P. 323 ff. (Marcel Decker Inc., New York, Basel, Hong Kong, edited by: Jürgen Fuchs, Frankfurt, and Lester Packer, Berkeley/California) deals with oxidative damage to the skin and its more immediate causes.

Additional antioxidants and/or radical interceptors can be incorporated in cosmetic or dermatological formulations in order to prevent such reactions.

Antioxidants and radical interceptors are of course well-known. The use of vitamin E, a substance with well-known antioxidant effects in light protection formulations, has therefore already been proposed in the U.S. Pat. Nos. 4,144,325 and 4,248,861 as well as in numerous other documents, but the effect attained by this remains far behind what had been hoped for.

### SUMMARY OF THE INVENTION

It was therefore the task of the present invention to find ways of countering the drawbacks of the prior art. In particular, the effect of the remedy for the damage associated with the endogenous, chronological and exogenous skin ageing should be permanent and sustainable prophylaxis without the risk of side effects.

However, it was surprising and unforeseeable to the skilled expert that cosmetic or dermatological preparations with a content of the active agent combination comprising creatinine and/or derivatives thereof with creatine and/or derivatives thereof as well as vegetable and animal extracts containing the same and soybean germ extracts or ingredients which can be isolated from this, remedies the disadvantages of the prior art.

On application of the active agent combination used according to the invention or cosmetic or topical dermatological preparations with an effective content of the active agent combination according to the invention, it was surprisingly possible to achieve an effective treatment and prophylaxis of:
deficient, sensitive or hypoactive skin conditions or deficient, sensitive or hypoactive conditions of cutaneous appendages;
symptoms of premature ageing of the skin (e.g. wrinkles, age marks, telangiectasias) or of the cutaneous appendages;
environmentally-induced (smoking, smog, reactive oxygen species, free radicals) and, in particular, light-induced negative changes to the skin and the cutaneous appendages;
light-induced skin damage;
pigmentation disorders;
itchiness;
dry skin conditions and stratum corneum disorders; hair loss and for improved hair growth; and
inflammatory skin conditions as well as atopic eczema, seborrhoeic eczema, polymorphous light dermatosis, psoriasis, vitiligo.

The active agent combination according to the invention or cosmetic or topical dermatological preparations with an effective content of the active agent combination corresponding to the invention also surprisingly serve to:
sooth sensitive or irritated skin;
stimulate collagen, hyaluronic acid, elastin synthesis;
stimulate intracellular DNA synthesis, in particular with deficient or hypoactive skin conditions
increase cell renewal and regeneration of the skin; and
increase the skin's own protective and repair mechanisms (e.g. for dysfunctional enzymes, DNA, lipids, proteins); as well as for preliminary and subsequent treatment during the topical application of laser and abrasion treatments, which for instance serve to reduce skin wrinkles and scars, in order to counteract the resultant skin irritations and promote the regeneration process for injured skin.

### DETAILED DESCRIPTION OF THE PREFERRED EMBODIMENTS

It is preferred if the content of creatinine and derivatives thereof is 0.001-50% by weight, preferably 0.1-10% by weight, and if the content of creatine and its derivatives is 0.001-50% by weight, preferably 0.1-10% by weight, in relation to the total weight of the preparations.

It is especially preferred if daidzin, glycitin, genistin, daidzein, glycitein, genistein and saponins are used as ingredients from soybean germ extracts.

It is preferred if the preparations contain 0.1 to 20% by weight, preferably 0.5 to 10% by weight, especially preferred 1 to 5% by weight soybean germ extracts or 0.01 to 2% by weight, preferably 0.05 to 1% by weight, especially preferred 0.1 to 0.55% by weight ingredients which can be isolated from this.

Preparations according to the invention are preferably used for restructuring and/or rejuvenating the skin, for alleviating cicatrization with reduced scar formation in the event of injuries to the epidermis as well as for inspiration of the body and senses.

The invention also comprises the use of such preparations for the prophylaxis or treatment of inflammatory skin conditions or for skin protection with sensitively determined and dry skin (such as atopic eczema, seborrhoeic eczema, polymorphous light dermatosis, psoriasis, vitiligo, wound healing disorders, itchiness, sensitive or irritated skin, light-induced skin damage and UV-induced immunosuppression, desquamation changes, changes in the normal fibroblast and ceratinocyte proliferation, changes in the normal fibroblast and ceratinocyte differentiation of deficient, sensitive or hypoactive skin conditions or deficient, sensitive or hypoactive conditions of the cutaneous appendages and for reduction of the skin thickness).

The invention also encompasses the use of such preparations for the treatment and prophylaxis of the symptoms of intrinsic or extrinsic skin ageing as well as for the treatment and prophylaxis of the harmful effects of ultraviolet radiation on the skin (such as degenerative appearances of the skin (typically age marks, wrinkles, telangiectasias, skin flabbiness, loss of elasticity, as well as the atrophy of epidermal and dermal cell layers, the components of the connective tissue, the rete pegs and capillary vessels), so-called skin luster and fatigue (skin limpness or skin tiredness), increased activation of proteolytic enzymes in the skin as well as, for example, metalloproteinases, disorders in the normal collagen, hyaluronic acid, elastin and glycosaminoglycan homeostasis and normal skin regeneration, changes in the normal fibroblast and ceratinocyte proliferation, changes in the normal fibroblast and ceratinocyte differentiation, deficiency symptoms of intracellular DNS synthesis (in particular with deficient or hypoactive skin deficiencies), environmentally-induced negative changes to the skin or the cutaneous appendages (caused by smoking, smog, reactive oxygen species, free radicals and similar).

The invention also encompasses the use of such preparations for the treatment and/or prophylaxis of pigmentation disorders, for enhancing ceramide biosynthesis (such as changes in the ceramide, lipid and energy source metabolism of healthy skin), for strengthening the barrier function of the skin (for example stratum corneum barrier disorders, changes in the normal lipid peroxidations, changes in the transepidermal water loss and the normal moisture content of the skin), for the treatment and/or prophylaxis of disorders concerning the normal skin pH value and the osmolyte balance, for the treatment and/or prophylaxis of divergences from the normal cell-cell communication in the skin (e.g. intercellular communication via mediators or via mechanical/physiological connections).

The invention also encompasses the use of such preparations for the treatment and/or prophylaxis of functional disorders to the cutaneous appendages (e.g. hair loss, improved hair growth, seborrhoeic symptoms, greasy skin, greasy hair, comedones, and also dandruff).

Effects corresponding to the invention are represented by the stabilization of the energy source metabolism in the sense of a synergism between the components according to the invention creatine, creatinine and soybean germ extracts, in comparison to creatine and creatinine alone. The improved energy situation of the skin cells enables a significantly better effect to be attained for the prophylaxis and treatment of degenerative skin and hair symptoms (wrinkles, fatigue, degeneration, age marks, circulatory disorders, itchiness, sensitivity to stress, brittleness).

Creatinine (from τO κρεαç=“flesh”) is characterized by the following

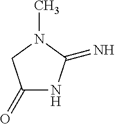

and is formed in the organism through non-enzymatic transformation from creatine phosphate corresponding to

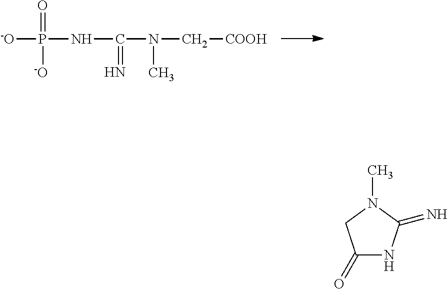

It is excreted via the kidneys. The level of creatinine excretion is proportional to the muscle mass and almost constant for the respective individual. Creatinine is present in meat extract and bouillon cubes.

Creatine (also from Greek: τO κρεαç=“flesh”) is characterized by the following structure:

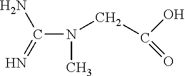

It is to be found in the muscular liquid of vertebrates at 0.05-0.4%, in small amounts also in the brain and blood. As a monohydrate, it represents a colorless, crystalline powder. Creatinine is formed in aqueous solution. In organisms it is formed through transamidination of L-arginine on glycine to guanidine acetic acid and the subsequent methylation of the latter by means of S-adenosylmethionine (via guanidine acetate methyltransferase). Creatine is regarded as an appetizing component of beef and meat extract. Creatine additive in food increases physical performance.

It is advantageously preferred to select the weight ratio of creatinine to creatine from the region between 50:1 and 1:50, preferably from 10:1 to 1:10, especially preferred from 2:1 to 1:2.

The preferred derivative is creatine phosphate, which exhibits the following structure:

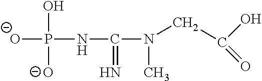

and is located in new muscle tissue where it plays an important role as an energy-storing phosphate (phosphagen). When the muscle is working, creatine phosphate with adenosine-5′-diphosphate produces adenosine-5′-triphosphate (ATP) and creatine under the influence of the enzyme creatine kinase; the reverse reaction occurs in muscle in a restive state.

However, creatine sulfate, creatine acetate, creatine ascorbate and the derivates esterified on the carboxyl group with mono- or polyfunctional alcohols also yield advantageous embodiments of the invention.

Soybean germ extracts which contain, amongst other ingredients, daidzin, glycitin, genistin, daidzein, glycitein, genistein as well as saponines are typically available commercially from the company L.M Cosmetics, Thiais, France with the designation isoflavone 150 (also referred to as isoflavone 150 in the following). This mixture also contains, for example, 39.01 ppm daidzin, 1.53 ppm malonyldaidzin, 20.56 ppm acetyldaidzin, 22.47 ppm glycitin, 1.74 ppm malonylglycitin, 12.69 ppm acetyiglycitin, genistin, 1.43 ppm daidzein, 9.82 ppm glycitein, 9.82 ppm genistin, 0.1 ppm malonylgenistin, 5.7 ppm acetylgenistin, 0.33 ppm genistein.

It is extremely advantageous in accordance with the invention to use the active agent combination corresponding to the invention, or cosmetic or dermatological preparations with an effective content of an active agent combination corresponding to the invention for the cosmetic or dermatological treatment or prophylaxis of undesired skin conditions.

Within the sense of the invention, standard antioxidants can be added to preparations which contain the active agent combinations corresponding to the invention.

It is advantageous to select the antioxidants from the group consisting of amino acids (e.g. glycine, histidine, tyrosins, tryptophan) and derivatives thereof, imidazoles (e.g. urocanic acid) and derivatives thereof, peptides such as D,L-carnosine, D-carnosine, L-carnosine and derivatives thereof (e.g. anserin), carotenoides, carotenes (e.g. α-carotene, β-carotene, lycopene) and derivatives thereof, Lipoic acid and derivatives thereof (e.g. dihydrolipoic acid), aurothio-glucose, propylthiouracil and other thiols (e.g. thio-redoxin, glutathione, cystein, cystine, cystamine and their glycosyl-, N-acetyl-, methyl-, ethyl-, propyl-, amyl-, butyl- and lauryl-, palmitoyl-, oleyl-, γ-linoleyl-, cholesteryl- and glyceryl esters) and salts thereof, di-lauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, butionine sulfones, penta-, hexa- and heptathionine sulfoximine) in very low tolerable doses (e.g. pmol to μmol/kg), furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alanine diacetic acid, flavonoids, polyphenols, catechins, vitamin C and derivatives (e.g. ascorbyl palmitate, Mg-ascorbyl phosphate, ascorbyl acetate), tocopherols and derivatives (e.g. vitamin E acetate), and coniferyl benzoate of benzoin resin, rutic acid and derivatives thereof, ferulic acid and derivatives thereof, butyl-hydroxytoluene, butylhydroxyanisole, nordihydro-guaiaretic acid, nordihydroguaiaretic acid, trihydroxy-butyrophenone, uric acid and derivatives thereof, mannose and derivatives thereof, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives suitable according to the invention (salts, esters, ethers, sugar, nucleotides, nucleosides, peptides and lipids) of this active agent cited.

The amount of antioxidants (one or more compounds) in the preparations is preferably 0.001 to 30% by weight, especially preferred 0.05-20% by weight, in particular 1-10% by weight, in relation to the total weight of the preparation.

Prophylaxis and/or cosmetic or dermatological treatment with the active agent combinations used corresponding to the invention, or with the cosmetic or topical dermatological preparations with an effective content of an active agent combination used corresponding to the invention, is realized in the standard manner, furthermore in such a way that the active agent used corresponding to the invention, or the cosmetic or topical dermatological preparations with an effective content of an active agent used corresponding to the invention, are applied to the areas affected on the skin.

The active agent combination according to the invention can advantageously be incorporated in the usual cosmetic and dermatological preparations which can be present in various forms. They can therefore represent, for example, a solution, an emulsion of the type water-in-oil (W/O) or of the type oil-in-water (O/W), or a multiple emulsion, for example of the type water-in-oil-in-water (W/O/W) or oil-in-water-in-oil (O/W/O), a hydrodispersion or lipodispersion, a gel, a solid pen or also an aerosol.

Emulsions in the sense of the present invention, e.g., in the form of a cream, a lotion, a cosmetic milk, are advantageous and contain, for example, fats. oils, waxes or other fat bodies, as well as water and one or more emulsifiers, as are typically used for such a type of formulation.

It is also possible in the sense of the present invention to add the active agent combination used corresponding to the invention to aqueous systems or surfactant preparations for cleaning the skin and hair.

The skilled expert is, of course, aware that high-quality cosmetic compounds are generally inconceivable without the usual auxiliary agents and additives. These include, for example, consistency providers, fillers, perfume, dyes, emulsifiers, additional active agents such as vitamins or proteins, sunscreen agents, stabilizers, insect repellants, alcohol, water, salts, substances with antimicrobial, proteolytical or keratolytical effect etc.

Mutatis mutandis, corresponding requirements apply in respect to the formulation for medical preparations.

Medical topical compounds in the sense of the present invention generally contain one or more medicines in an effective concentration. For the sake of simplicity, attention is drawn to the statutory provisions of the Federal Republic of Germany (e.g. Cosmetics Ordinance, Food and Medical Drugs Law) for proper distinction between cosmetic and medical applications and corresponding products.

It is also advantageous to add the active agent combination corresponding to the invention to preparations which already contain other active agents for other purposes.

Accordingly, cosmetic or topical dermatological compounds in the sense of the present invention can also be used, for example, as skin protection care cream, cleaning milk, sun protection lotion, replenishing cream, day or night cream etc, depending on their structure. It is potentially possible and advantageous to use the compounds corresponding to the invention as the basis for pharmaceutical formulations.

Such cosmetic and dermatological preparations which are present in the form of a sunscreen are also potentially favorable. These preferably contain at least one UVA filter substance and/or at least one UVB filter substance and/or at least one inorganic pigment in addition to the active agent combination corresponding to the invention.

It is, however, advantageous in the sense of the present inventions to create those such cosmetic and dermatological preparations whose primary purpose is not protection against sunlight but which, however, exhibit a content of UV protection substances. UV-A or UV-B filter substances are therefore usually incorporated in day creams for example.

Preparations corresponding to the invention can advantageously contain substances which absorb the UV radiation in the UVB range, whereby the total amount of filter substances is, e.g, 0.1 to 30% by weight, preferably 0.5 to 10% by weight, in particular 1 to 6% by weight, in relation to the total weight of the preparations.

The UVB filters can be oil-soluble or water-soluble. Typical oil-soluble substances include:
3-benzylidene camphor and derivatives thereof, e.g. 3-(4-methylbenzyliden)camphor,
4-aminobenzoic acid derivatives, preferably 4-(dimethylamino)-benzoic acid(2-ethylhexyl)ester, 4-(dimethylamino)benzoic acid amylester; esters of cinnamic acid, preferably 4-methoxy cinnamic acid(2-ethylhexyl)ester, 4-methoxy cinnamic acid isopentylester;
esters of salicylic acid, preferably salicylic acid(2-ethylhexyl)ester, salicylic acid(4-isopropylbenzyl)ester, salicylic acid homomenthylester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzalmalonic acid di(2-ethylhexyl)ester; and
2,4,6-trianilino-(p-carbo-2′-ethyl-l′-hexyloxy)-1,3,5-triazine.

Advantageous as water-soluble substances are:
2-phenylbenzimidazol-5-sulfonic acid and salts thereof, e.g. sodium, potassium or triethanolammonium salt;
sulfonic acid derivatives of benzophenones, preferably 2-hydroxy-4-methoxybenzophenon-5-sulfonic acid and salts thereof; and
sulfonic acid derivatives of 3-benzylidene camphor, such as 4-(2-oxo-3-bornylidene methyl)benzolsulfonic acid, 2-methyl-5-(2-oxo-3-bornylidene methyl)sulfonic acid and salts thereof.

The list of the UVB filters indicated which can be used according to the invention should, of course, not be restrictive.

The object of the invention is also the combination of a UVA filter corresponding to the invention with a UVB filter or a cosmetic or dermatological preparation corresponding to the invention which also contains a UVB filter.

It can also be advantageous to use UVA filters in preparations corresponding to the invention, which are normally contained in cosmetic and/or dermatological preparations. Such filter substances preferably involve derivatives of dibenzoyl methane, in particular 1-(4′-tert.butylphenyl)-3-(4′-methoxyphenyl)propane-1,3-dione and 1-phenyl-3-(4′-isopropylphenyl) propane-l,3-dione. Preparations which contain these combinations are also the object of the invention. The same amounts of UVA filter substances can be used as those which have been cited for UVB filter substances.

Cosmetic or dermatological preparations in the sense of the present invention can also contain inorganic pigments which are normally used in cosmetics to protect the skin form UV radiation. These involve oxides of titanium, zinc, iron, zirconium, silicon, manganese, aluminum, cerium and mixtures thereof, as well as modifications in which the oxides are the active agents. Particularly preferred are pigments based on titanium dioxide. The amounts indicated for the above combinations can be used.

The cosmetic and dermatological preparations corresponding to the invention may contain cosmetic, active, auxiliary and/or additive agents, as they are normally used in such preparations, e.g., antioxidants, preservatives, bactericides, perfumes, substances to prevent foaming, dyes, pigments which have a coloring effect, thickening agents, surfactants, emulsifiers, moisturizing and/or moisture-retaining substances, fats, oils, waxes or other standard components of a cosmetic or dermatological formulation such as alcohols, polyols, polymers, foam stabilizers, electrolytes, organic solvents or silicone derivatives.

Insofar as the cosmetic or dermatological preparation in the sense of the present invention represents a solution or emulsion or dispersion, the following can be used as solvents:
Water or aqueous solutions
Oils, such as triglycerides of capric or caprylic acid, preferably however castor oil; fats, waxes and other natural and synthetic fat bodies, preferably esters of fatty acids with alcohols exhibiting a low C-number, e.g. with isopropanol, propylene glycol or glycerol, or esters of fatty alcohols with alcanoic acids exhibiting a low C-number or with fatty acids; and alcohols, diols or polyols of a low C-number, as well as ethers thereof, preferably ethanol, isopropanol, propylene glycol, glycerol, ethylene glycol, ethylene glycolmonoethyl- or -monobutylether, propylene glycolmonomethyl, -monoethyl- or -monobutylether, diethylene glycolmonomethyl- or -monoethylether and analogous products.

Mixtures of the solvents indicated above are used in particular. With alcoholic solvents water can be a further component.

The oil phase of the emulsions, oleogels, hydrodispersions or lipodispersions in the sense of the present invention is preferably selected from the group of esters comprising saturated and/or unsaturated, branched and/or unbranched alkane carboxylic acids with a chain length of 3 to 30 C atoms and saturated and/or unsaturated, branched and/or unbranched alcohols with a chain length of 3 to 30 C atoms, from the group of esters comprising carboxylic acids and saturated and/or unsaturated, branched and/or unbranched alcohols with a chain length of 3 to 30 C atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate as well as synthetic, semi-synthetic and natural mixtures of such esters, e.g. jojoba oil.

The oil phase can also advantageously be selected from the group of branched and unbranched hydrocarbons and waxes, the silicone oils, the dialkyl ethers, the group of saturated or unsaturated, branched or unbranched alcohols, as well as the fatty acid triglycerides, particularly the triglycerin esters of saturated and/or unsaturated, branched and/or unbranched alkane carboxylic acids with a chain length of 8 to 24, in particular 12-18 C atoms. The fatty acid triglycerides can, for example, advantageously be selected from the group of synthetic, semi-synthetic and natural oils, e.g. olive oil, sunflower oil, soybean oil, peanut oil, rapeseed soil, almond oil, palm oil, coconut oil, palm-kernel oil and similar.

Any blends of such oil and wax components can also be used advantageously in the sense of the present invention. It may also be advantageous to use waxes, for example cetyl palmitate, as sole lipid components of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/caprinic acid triglyceride, dicaprylyl ether.

Especially advantageous are mixtures comprising C₁₂₋₁₅-alky benzoate and 2-ethylhexyl isostearate, mixtures comprising C₁₂₋₁₅-alky benzoate and isotridecyl isononanoate as well as mixtures comprising C₁₂₋₁₅-alkyl benzoate, 2-ethylhexyl isostearate and isotridecyl isononanoate.

Among the hydrocarbons, paraffin oil, squalene and squalene are to be used advantageously in the sense of the present invention.

The oil phase can also advantageously exhibit a content of cyclic or linear silicone oils or completely consist of such oils, whereby it is nevertheless preferred to use an additional content of other oil phase components apart from silicone oil or silicone oils.

Cyclomethicone (octamethylcyclotetrasiloxane) can be used advantageously as a silicone oil according to the invention. However, other silicone oils are also to be used advantageously in the sense of the present invention, for example hexamethylcyclotrisiloxane, polydimethylsiloxane, poly(methylphenylsiloxane). Mixtures comprising cyclomethicon and isotridecyl isononanoate, or cyclomethicon and 2-ethylhexyHsostearate are particularly advantages.

The aqueous phase of the preparations corresponding to the invention may advantageously contain alcohols, diols or polyols with a low C number, as well as ethers thereof, preferably ethanol, isopropanol, propylene glycol, glycerol, ethylene glycol, ethylene glycol monoethyl- or -monobutylether, propylene glycol monomethyl, monoethyl- or -monobutylether, diethylene glycolmonomethyl- or -monoethylether and analogous products, as well as alcohols with a low C number, e.g. ethanol, isopropanol, 1,2-propane dial, glycerol as well as, in particular, one or more thickening agents, which can advantageously be selected from the group silicon dioxide, aluminum silicates, polysaccharides or derivatives thereof, e.g. hyaluronic acid, Xanthan gum, hydroxypropylmethyl cellulose, particularly advantageously from the group of polyacrylates, preferably a polyacrylate from the group of so-called carbopols, for example carbopols of the types 980, 981, 1382, 2984, 5984, either individually or in combination.

Gels used in the sense of the present invention usually contain alcohols of a low C number, e.g. ethanol, isopropanol, 1,2-propane diol, glycerol and water or an abovementioned oil in the presence of a thickening agent which for oily-alcoholic gels is preferably silicon dioxide or an aluminum silicate, or which is preferably a polyacrylate for aqueous-alcoholic or alcoholic gels.

Solid pens contain, for example, natural or synthetic waxes, fatty alcohols or fatty acid esters.

The usual elements which are suitable for use as cosmetic pens in the sense of the present invention are liquid oils (e.g. paraffin oils, castor oil, isopropyl myristate), semisolid components (e.g. Vaseline, lanolin), solid components (e.g. beeswax, ceresin and microcrystalline waxes or ozocerite) as well as high-melting waxes, e.g. carnauba wax, candelilla wax)

The universally familiar, highly-volatile, liquefied propellants, for example hydrocarbons (propane, butane, isobutene), are suitable as propellants for cosmetic or dermatological preparations which can be sprayed from aerosols in the sense of the present invention. These can be used alone or mixed with each other. Compressed air can also be used advantageously.

The skilled expert is, of course, aware that there are non-toxic aerosol propellants which would be suitable for realizing the present invention in the form of aerosol preparations, but which should be dispensed with on account of the negative effects on the environment or other associated circumstances, in particular fluorinated hydrocarbons and chlorofluorocarbons (CFCs).

Cosmetic preparations in the sense of the present invention can also be present as gels which, in addition to an effective content of the active agent corresponding to the invention and solvents usually used for this purpose, preferably contain water, as well as organic thickening agents, e.g., gum arabic, Xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickening agents, e.g. aluminum silicates such as bentonites, or a mixture comprising polyethylene glycol and polyethylene glycol stearate or -distearate. An amount of thickening agent between 0.1 and 30% by weight, preferably between 0.5 and 15% by weight, is typically contained in the gel.

The following examples are intended to clarify the present invention.

### EXAMPLES

1. PIT Emulsions

The fat and water phases are heated separately to 80° C. in the laboratory scale. The fat phase is presented. At 80° C. the perfume is added, the water phase is then added. The emulsion is cooled to room temperature while being stirred. A homogenization is not necessary on account of the spontaneous formation of the emulsion.

| | Examples | | | | |
|---|---|---|---|---|---|
| | 1 | 2 | 3 | 4 | 5 |
| Glycerin monostearate, self- | 0.50 | | 3.00 | 2.00 | 4.00 |
| emulsifying | | | | | |
| Polyoxyethylene(12)cetylstearyl | | 5.00 | | 1.00 | 1.50 |
| ether | | | | | |
| Polyoxyethylene(20)cetylstearyl | | | | 2.00 | |
| ether | | | | | |
| Polyoxyethylene(20)cetylstearyl | 5.00 | | 1.00 | | |
| ether | | | | | |
| Stearyl alcohol | | | 3.00 | | 0.50 |
| Cetyl alcohol | 2.50 | 1.00 | | 1.50 | |
| 2-ethylhexyl methoxy cinnamate | | | | 5.00 | 8.00 |
| 2,4-bis-(4-(2-ethyl-hexyloxy-)2- | | 1.50 | | 2.00 | 2.50 |
| hydroxyl)-phenyl-6-(4-methoxyhenyl)- | | | | | |
| (1,3,5)-triazine | | | | | |
| 1-(4-tert-butylpheyl)-3-(4- | | | 2.00 | | |
| methoxyphenyl)-1,3-propane dione | | | | | |
| Diethylhexyl butamido triazone | 1.00 | 2.00 | | 2.00 | |
| Ethylhexyl triazone | 4.00 | | 3.00 | 4.00 | |
| 4-methylbenzylidene camphor | | 4.00 | | | 2.00 |
| Octocrylene | | 4.00 | | | 2.50 |
| Phenylene-l,4-bis-(monosodium, 2- | | | 0.50 | | 1.50 |
| benzimidazyl-5,7-disulfonic acid | | | | | |
| Phenylbenzimidazol sulfonic acid | 0.50 | | | 3.00 | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicon polydimethylsiloxane | | 0.50 | 1.00 | | |
| Phenylmethylpolysiloxane | 2.00 | | | 0.50 | 0.50 |
| Shea butter (Sheabutter) | | 2.00 | | | 0.50 |
| PVP hexadecane copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Isoflavone 150 | 0.10 | 0.20 | 0.20 | 0.50 | 0.10 |
| Creatinine | 0.30 | 0.10 | 0.60 | 0.20 | 0.30 |
| Creatine | 0.30 | 0.10 | 0.60 | 0.20 | 0.30 |
| Alpha-glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad.100 | ad.100 | ad.100 | ad.100 | ad.100 |

2. OM Cream

| | Examples | | | | |
|---|---|---|---|---|---|
| | 1 | 2 | 3 | 4 | 5 |
| Glyceryl stearate citrate | | | 2.00 | | 2.00 |
| Glyceryl sterate, self-emulsifying | 4.00 | 3.00 | | | |
| PEG-40-stearate | 1.00 | | | | |
| Polyglyceryl-3-methylglucose-distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/Capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-methylbenzylidene camphor | | | 1.00 | | |
| 1-(4-tert-butylphenyl)-3-(4- | | | 0.50 | | |
| methoxyphenyl)-1,3-propane dione | | | | | |
| Isoflavone 150 | 0.10 | 0.20 | 0.70 | 0.15 | 1.00 |
| Creatine | 0.30 | 0.30 | 0.50 | 0.10 | 1.00 |
| Creatinine | 0.30 | 0.30 | 0.50 | 0.10 | 1.00 |
| Tocopherol | 0.1 | | | | 0.20 |
| Biotin | | | 0.05 | | |
| Ethylene diamine tetraacetic acid | 0.1 | | 0.10 | 0.1 | |
| trisodium | | | | | |
| Preservative | q.s- | q.s. | ¹_q.s. | q.s. | q.s. |
| Polyacrylic acid | 3.00 | 0.1 | | 0.1 | 0.1 |
| Sodium hydroxide solution 45% | q.s | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | Ad 100 | Ad 100 | Ad 100 | Ad 100 |

| | Examples | | | | |
|---|---|---|---|---|---|
| | 6 | 7 | 8 | 9 | 10 |
| Glyceryl stearate citrate | | 2.00 | 2.00 | | |
| Glyceryl sterate, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetylstearyl alcohol | | 3.00 | 1.00 | | 0.50 |
| C12-15 alkyl benzoate | | 2.00 | 3.00 | • | |
| Caprylic/Capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzyliden Campher | 1.00 | | | | 1.00 |
| 1-(4-tert-Butylphenyl)-3-(4- | 0.50 | | | | 0.50 |
| methoxyphenyl)-1,3-propandione | | | | | |
| Isoflavone 150 | 0.30 | 0.10 | 1.00 | 0.50 | 0.10 |
| Creatinine | 0.10 | 0.30 | 0.20 | 0.10 | 0.20 |
| Creatine | 0.10 | 0.30 | 0.20 | 0.10 | 0.20 |
| Tocopherol | | | | | 0.05 |
| Ethylene diamine tetraacetic acid | | | 0.20 | | 0.20 |
| trisodium | | | | | |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | Ad 100 | Ad 100 | Ad 100 | Ad 100 | Ad 100 |

3. W/O Emulsions

| | Examples | | | | |
|---|---|---|---|---|---|
| | 1 | 2 | 3 | 4 | 5 |
| Cetyl dimethicone copolyol | | 2.50 | | 4.00 | |
| Polyglyceryl-2-dipolyhydroxystearate | 5.00 | | | | 4.50 |
| PEG-30-dipolyhydroxystearate | | | 5.00 | | |
| 2-ethylhexyl methoxy cinnamate | | 8.00 | | 5.00 | 4.00 |
| 2,4-bis-(4-(2-ethyl-hexyloxy-)2- | 2.00 | 2.50 | | 2.00 | 2.50 |
| hydroxyl)-phenyl)-6-(4- | | | | | |
| methoxyphenyl)-(1,3,5)-triazine | | | | | |
| 1-(4-tert-butylphenyl)-3-(4- | | | 2.00 | 1.00 | |
| methoxyphenyl)-1,3-propane dione | | | | | |
| Diethylhexyl butamido triazone | 3.00 | 1.00 | | | 3.00 |
| Ethylhexyl triazone | | | 3.00 | 4.00 | |
| 4-methylbenzylidene camphor | | 2.00 | | 4.00 | 2.00 |
| Octocrylene | 7.00 | 2.50 | 4.00 | | 2.50 |
| Diethylhexyl butamido triazone | 1.00 | | | 2.00 | |
| Phenylene-1,4-bis-(monosodium, 2- | 1.00 | 2.00 | 0.50 | | |
| benzimidazyl-5,7-disulfonic acid) | | | | | |
| Phenylbenzimidazol sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerin | | 0.30 | 1.00 | | 0.50 |
| Glycerin | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Isoflavone 150 | 0.10 | 0.20 | 0.50 | 0.90 | 1.00 |
| Creatinine | 0.10 | 0.30 | 0.20 | 0.40 | 0.30 |
| Creatine | 0.10 | 0.30 | 0.20 | 0.40 | 0.30 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad.100 | ad.100 | ad.100 | ad.100 | ad.100 |

Examples

| | 6 | 7 |
|---|---|---|
| Polyglyceryl-2-dipolyhydroxystearat | 4.00 | 5.00 |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| 1-(4-tert-butylphenyl)-3-(4-methoxyphenyl)- | 0.50 | 1.50 |
| 1,3-propane dione | | |
| 4-methylbenzylidene camphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerin | | 3.00 |
| Glycerol | 5.00 | |
| Tocopherol acetate | 0.50 | 1.00 |
| Isoflavone 150 | 0.10 | 0.70 |
| Creatinine | 1.00 | 0.60 |
| Creatine | 1.00 | 0.60 |
| Trisodium EDTA | 0.20 | 0.20 |
| Preservative | q.s. | q.s. |
| Ethanol | | 3.00 |
| Perfume | q.s. | q.s. |
| Water | ad. 100 | ad. 100 |

4. Hydrodispersions

| | Examples | | | | |
|---|---|---|---|---|---|
| | 1 | 2 | 3 | 4 | 5 |
| Polyoxyethylene(20)cetylstearyl ether | 1.00 | | | 0.5 | |
| Cetyl alcohol | | | 1.00 | | |
| Sodium polyacrylate | | 0.20 | | 0.30 | |
| Acrylate/C10-30 alkyl acrylate | 0.50 | | 0.40 | 0.10 | 0.10 |
| crosspolymer | | | | | |
| Xanthan gum | | 0.30 | 0.15 | | 0.50 |
| 2-ethylhexyl methoxy cinnamate | | | | 5.00 | 8.00 |
| 2,4-bis-(4-(2-ethyl-hexyloxy-)2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-butylphenyl)-3-(4- | 1.00 | | 2.00 | | |
| methoxyphenyl)-1,3-propane dione | | | | | |
| Diethylhexyl butamido triazone | | 2.00 | | 2.00 | 1.00 |
| Ethylhexyl triazone | 4.00 | | 3.00 | 4.00 | |
| 4-methylbenzylidene camphor | 4.00 | 4.00 | | | 2.00 |
| Octocrylene | | 4.00 | 4.00 | | 2.50 |
| Phenylene-1,4-bis-(monosodium, 2- | 1.00 | | 0.50 | | 2.00 |
| benzimidazyl-5,7-disulfonic acid | | | | | |
| Phenylbenzimidazol sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecane copolymer | 0.50 | | | 0.50 | 1.00 |
| Octoxyglycerin | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Isoflavone 150 | 0.3 | 0.10 | 0.50 | 0.30 | 0.20 |
| Creatinine | 0.10 | 0.30 | 1.00 | 0.70 | 0.50 |
| Creatine | 0.10 | 0.30 | 1.00 | 0.70 | 0.50 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad. 100 | ad.100 | ad.100 | ad. 100 | ad.100 |

5. Example (Gel Cream):

| | |
|---|---|
| Acrylate/C10-30 alkyl acrylate | 0.40 |
| crosspolymer | |
| Polyacrylic acid | 0.20 |
| Xanthan gum | 0.10 |
| Cetearyl alcohol | 3.00 |
| C12-15 alkyl benzoate | 4.00 |
| Caprylic/Capric triglyceride | 3.00 |
| Cyclic dimethylpolysiloxane | 5.00 |
| Dimeticone polydimethylsiloxane | 1.00 |
| Isoflavone 150 | 0.10 |
| Creatinine | 0.30 |
| Creatine | 0.30 |
| Glycerol | 3.00 |
| Sodium hydroxide | q.s. |
| Preservative | q.s. |
| Perfume | q.s. |
| Water | ad 100.0 |

pH value set to 6.0

6. Example (W/O Cream)

| | |
|---|---|
| Polyglyceryl-3-diisostearate | 3.50 |
| Glycerol | 3.00 |
| Polyglyceryl-2-dipolyhydroxystearate | 3.50 |
| Isoflavone 150 | 0.20 |
| Creatinine | 0.10 |
| Creatine | 0.10 |
| Preservative | q.s. |
| Perfume | q.s. |
| Water | ad 100.0 |
| Magnesium sulphate | 0.6 |
| Isopropyl stearate | 2.0 |
| Caprylyl ether | 8.0 |
| Cetearyl isononanoate | 6.0 |

7. Example (W/O/W Cream):

| | |
|---|---|
| Glyceryl stearate | 3.00 |
| PEG-100 stearate | 0.75 |
| Behenyl alcohol | 2.00 |
| Caprylic/Capric triglyceride | 8.0 |
| Octyldodecanol | 5.00 |
| C12-15 alkyl benzoate | 3.00 |
| Isoflavone 150 | 1.00 |
| Creatinine | 1.00 |
| Creatine | 1.00 |
| Magnesium sulfate (MgSO₄) | 0.80 |
| Ethylene diamine tetraacetic acid | 0.10 |
| Preservative | q.s. |
| Perfume | q.s. |
| Water | ad 100.0 |

pH value set to 6.0

What is claimed is:

1. A method of restructuring or rejuvenating skin, wherein the method comprises applying to the skin a cosmetic or dermatological preparation comprising: (a) at least one compound selected creatinine and derivatives of creatinine; (b) at least one compound selected from creatine and derivatives of creatine; and (c) at least one substance selected from soybean germ extracts and ingredients which can be isolated from soybean germ.

2. A method of stimulating collagen synthesis of skin, wherein the method comprises applying to the skin a cosmetic or dermatological preparation comprising: (a) at least one compound selected from creatinine and derivatives of creatinine; (b) at least one compound selected from creatine and derivatives of creatine; and (c) at least one substance selected from soybean germ extracts and ingredients which can be isolated from soybean germ.

3. The method of claim 2, wherein a three-dimensional structure of a dermis/epidermis juncture is improved.

4. The method of claim 2, wherein scar formation is reduced.

5. A method for the treatment and/or prophylaxis of one or more of deficient, sensitive or hypoactive conditions of skin and/or cutaneous appendages; symptoms of premature aging of skin and/or cutaneous appendages; pigmentation disorders; itchiness; hair loss; atopic eczema; seborrheic eczema; psoriasis; and vitiligo, wherein the method comprises applying to skin in need of treatment and/or prophylaxis a cosmetic or dermatological preparation comprising: (a) at least one compound selected from creatinine and derivatives of creatinine; (b) at least one compound selected from creatine and derivatives of creatine; and (c) at least one substance selected from soybean germ extracts and ingredients which can be isolated from soybean germ.

6. The method of claim 5, wherein the preparation comprises, based on a total weight of the preparation:
from 0.1% to 10% by weight of (a) creatinine;
from 0.1% to 10% by weight of (b) at least one of creatine, creatine phosphate, creatine sulphate, creatine acetate, creatine ascorbate, and an ester of creatine and a mono- or polyfunctional alcohol; and
from 0.05% to 5% by weight of (c) a soybean germ extract which comprises one or more of daidzin, malonyldaidzin, acetyldaidzin, glycitin, malonylglycitin, acetylglycitin, genistin, daidzein, glycitein, malonylgenistin, acetylgenistin, genistein and a saponin.

7. The method of claim 6, wherein the preparation comprises at least one of daidzin, glycitin, genistin, daidzein, glycitein, and genistein.

8. The method of claim 6, wherein the preparation comprises from 0.1% to 1% by weight of (c).

9. The method of claim 5, wherein components (a), (b) and (c) are present in concentrations which render the preparation effective for the treatment and/or prophylaxis of pigmentation disorders.

10. The method of claim 5, wherein components (a), (b) and (c) are present in concentrations which render the preparation effective for the treatment and/or prophylaxis of itchiness.

11. The method of claim 5, wherein components (a), (b) and (c) are present in concentrations which render the preparation effective for the treatment and/or prophylaxis of atopic eczema.

12. The method of claim 5, wherein components (a), (b) and (c) are present in concentrations which render the preparation effective for the treatment and/or prophylaxis of seborrheic eczema.

13. The method of claim 5, wherein components (a), (b) and (c) are present in concentrations which render the preparation effective for the treatment and/or prophylaxis of psoriasis.

14. The method of claim 5, wherein components (a), (b) and (c) are present in concentrations which render the preparation effective for the treatment and/or prophylaxis of vitiligo.

15. The method of claim 5, wherein the preparation comprises components (a), (b) and (c) in concentrations which render the preparation additionally capable of soothing sensitive or irritated skin.

16. The method of claim 5, wherein the preparation comprises components (a), (b) and (c) in concentrations which render the preparation additionally capable of stimulating collagen, hyaluronic acid and elastin synthesis.

17. The method of claim 5, wherein the preparation comprises components (a), (b) and (c) in concentrations which render the preparation additionally capable of stimulating intracellular DNA synthesis.

18. The method of claim 5, wherein the preparation comprises components (a), (b) and (c) in concentrations which render the preparation additionally capable of treatment and/or prophylaxis of disorders related to skin pH and/or osmolytic balance.

19. The method of claim 5, wherein the preparation comprises components (a), (b) and (c) in concentrations which render the preparation additionally capable of reducing scar formation of injured skin.

20. The method of claim 5, wherein the preparation comprises components (a), (b) and (c) in concentrations which render the preparation additionally capable of enhancing ceramide biosynthesis.
